(19) United States
(12) Patent Application Publication
(10) Pub. No.: US 2010/0016890 A1
(43) Pub. Date: Jan. 21, 2010

(54) Spool Dip And Overcoat Process For Medical Devices

(76) Inventors: Steve Tsai, Stamford, CT (US); Jon Reinprecht, Watertown, CT (US)

Correspondence Address: Mark Farber, Esq.;Tyco Healthcare Group LP, 60 Middletown Avenue, North Haven, CT, 06473, US

(21) Appl. No.: 12/499,858

(22) Filed: Jul. 9, 2009

Related U.S. Application Data
(60) Provisional application No. 61/081,520, filed on Jul. 17, 2008.

(51) Int. Cl. A61B 17/04; B05C 3/02; B05C 11/10; B05C 13/00

(52) U.S. Cl. 606/228; 118/400; 427/2.31; 118/416; 118/58

(57) ABSTRACT

A system and method for coating a suture are disclosed. The system includes a spool including a core having a suture wrapped thereabout and a dip tank including a first coating composition. The dip tank is configured to submerge the spool therein, thereby coating the suture with the first composition to form a pre-coated suture. The system also includes a coating device including a second coating composition. The coating device is configured to overcoat the pre-coated suture with the second coating composition.

### CROSS-REFERENCE TO RELATED APPLICATION

The present application claims the benefit of, and priority to, U.S. Provisional Patent Application Ser. No. 61/081,520 filed on Jul. 17, 2008, the entire disclosure of which is incorporated by reference herein.

### BACKGROUND

1. Technical Field

The present disclosure relates generally to systems and methods for coating medical devices, and in particular to systems and methods for coating sutures.

2. Description of the Related Art

Techniques for coating sutures are known. Coatings may benefit sutures by improving the strength or knot tie-down characteristics as well as by increasing surface lubricity, which in turn reduces the friction associated with passing the suture through tissue. Coatings may also provide therapeutic benefits to the tissue as a drug carrier.

Generally, coatings are applied by passing a suture line into or through a coating composition. Although this technique has been conventionally used to provide acceptable coatings for sutures, the suture must be unwound in order to pass the suture line through the coating. It would be advantageous to provide a method of coating sutures wound on a spool. Further, it would be advantageous to provide a method for coating a spool of suture.

### SUMMARY

A system and method for coating a suture are disclosed. The system includes a spool including a core having a suture wrapped thereabout and a dip tank including a first coating composition. The spool may have perforations along any length of the solid or hollow core and may include flanged ends. The suture is wrapped in a configuration to maximize the surface area of the suture that is exposed during coating. The dip tank is configured to fully submerge the spool therein, thereby coating the suture with the first composition to form a pre-coated suture. The first composition may include an active agent.

In embodiments, the system may also include a coating device including a second coating composition. The coating device is configured to overcoat the pre-coated suture with the second coating composition. The coating device may be, for example, a dip tank, a horizontal dip coater, a coating head, a filling head, a sprayer, or a dip coat syringe.

According to another embodiment of the present disclosure, a method for coating at least one suture is disclosed. The method includes providing a spool of suture including a core having a suture wrapped thereabout and dipping the spool into a first coating composition thereby forming a pre-coated suture on the spool. In embodiments, dipping includes soaking the spool of suture in the first coating composition. In embodiments, the spool of suture may be agitated in the first coating composition while soaking.

The method may also include draining the first coating composition and drying the spool of suture to remove excess amounts of the first coating composition. In embodiments, drying may be accomplished by spinning the spool of suture and/or drying the spool in a vacuum drying chamber. In embodiments, an integrated coating and drying tank system utilizing a tank having a rotational driver and shaft may be used to coat and spin dry a spool of suture. The method may further include coating the pre-coated suture with at least a second suture composition to form an overcoat on the pre-coated suture.

An integrated coating and spin drying tank system is also disclosed. The system includes a spool including a core having a suture wrapped thereabout and a dip tank including a first coating composition. The dip tank includes a rotational driver and a shaft configured to removably couple to the spool. The rotational driver is configured to spin the spool within the tank thereby coating the suture with the first composition and thereafter spin-drying the spool to form a pre-coated suture.

### BRIEF DESCRIPTION OF THE DRAWINGS

FIG. 1 is a perspective view of a spool dip system according to one embodiment of the present disclosure;

FIG. 2A is a perspective view of a spool according to one embodiment of the present disclosure;

FIG. 2B is a perspective view of a spool according to another embodiment of the present disclosure;

FIG. 2C is a perspective view of a spool according to a further embodiment of the present disclosure;

FIG. 3 is a cross-sectional front view of a spool dip system including a table shaker according to one embodiment of the present disclosure;

FIG. 4A is a cross-sectional side view of a spool dip system having a horizontal dip tank and rotating shaft according to one embodiment of the present disclosure;

FIG. 4B is a side view of the spool dip system of FIG. 4A along the line 4B according to one embodiment of the present disclosure;

FIG. 5A is a cross-sectional side view of a spool dip system having a vertical cylindrical tank and rotating shaft according to one embodiment of the present disclosure;

FIG. 5B is a top view of the spool dip system of FIG. 5A along the line 5B according to one embodiment of the present disclosure;

FIG. 6 is a schematic diagram of a horizontal dip coating system according to one embodiment of the present disclosure; and

FIG. 7 is a flow chart of a spool dip and overcoat process according to one embodiment of the present disclosure.

### DETAILED DESCRIPTION

Embodiments of the spool dip and overcoat process for coating a medical device of the present disclosure will now be described in detail with reference to the drawings wherein like reference numerals identify similar or like elements throughout the several views. Medical devices refer to articles which are useful for diagnostic and therapeutic purposes, particularly filamentous materials.

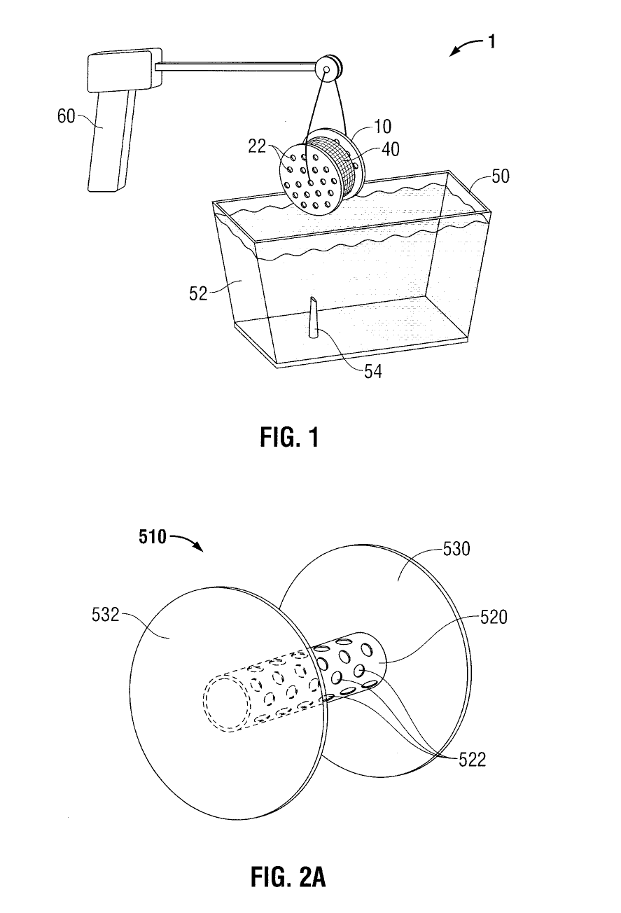

FIG. 1 illustrates spool dip system 1 for coating suture 40 wound on spool 10. System 1 includes tank 50 having coating composition 52. Any reservoir or vessel adapted to hold a volume of solution capable of submerging and wetting spool 10 of suture 40 therein may be utilized. In embodiments, tank 50 may be a rectangular, cylindrical, or any other shaped reservoir or vessel. Tank 50 may be made of stainless steel, titanium, plastics, glass, or other suitable materials capable of holding coating composition 52.

System 1 also includes dipping mechanism 60 which submerges spool 10 into dip tank 50. Spool 10 of suture 40 may be placed within dip tank 50 by various manual techniques or mechanical devices, such as, for example, a crane or other lifting and lowering apparatus, methods of which are within the purview of those skilled in the art. Further, dip tank 50 may contain support or post 54 to hold spool 10 of suture 40 in a pre-determined location within dip tank 50 to keep suture 40 from contacting the tank wall during shaking. There may be multiple posts 54 for multiple spools 10 for simultaneous dip coating.

Spool 10 may have a desired type and length of suture 40 wrapped thereabout. Suture 40 may be a monofilament, multi-filament, or braided suture fabricated from synthetic or natural materials, or combinations thereof. Suture types, configurations, and materials are dependent on the desired application of use as known to those skilled in the art.

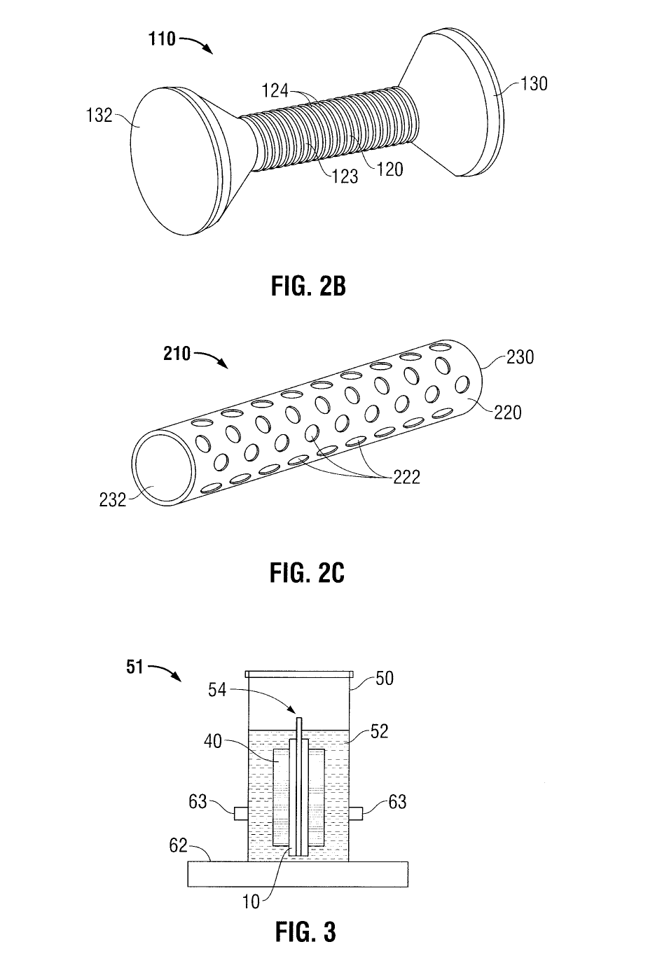

Spool 10 may be a reel, coil, bobbin, or any other apparatus adapted for holding sutures 40. For example, as illustrated in FIG. 2A, spool 510 has a substantially cylindrical core 520 and ends 530 and 532, which are shown as flanges having a diameter larger than the diameter of core 520 for maintaining suture 40 therebetween. Core 520 may be cylindrical or any other shape. In embodiments, core 520 may be solid or hollow. In embodiments, core 520 has perforations 522 to allow the coating to coat the suture closest to the spool center. Spool 510 has perforations 522 along a length of core 520 and may have perforated ends (as shown in FIG. 1) of any size and shape. Perforations 522 may be any shape including, but not limited to, circles, triangles, rectangles, rhombuses, pentagons, hexagons, octagons, ovals, other geometric shapes, and irregular shapes. Spool 510 may be formed of any suitable material compatible with suture 40 and the coating compositions utilized in the system. In some embodiments, spool 510 is stainless steel having a perforated core 520. Suture 40 may be cross-wound along core 520 to increase the exposed surface of suture 40 for maximum suture-liquid contact.

FIG. 2B illustrates another embodiment of the presently described spool shown generally as 110. Spool 110 includes core 120 and ends 130 and 132, which taper into core 120. The core 120 includes ribbed surface 123 having a plurality of threads 124 configured to retain sutures. FIG. 2C illustrates a further embodiment of spool 210 having a cylindrical shaped core 220 and ends 230 and 232 of a substantially consistent diameter with core 220. It should be understood that spools of the present disclosure may each be used interchangeably with different embodiments of spool dip systems of the present disclosure.

It is also contemplated that spool 10 may have any core 20 configuration allowing for a length of suture 40 to be wrapped thereabout for bulk dip coating as is within the purview of those skilled in the art. In embodiments, core 20 may include a plurality of wires arranged in a tubular manner (e.g., parallel helix configuration). In other embodiments, spool 10 may have an irregularly shaped core 20 including protrusion (not shown) raised therefrom and/or perforations 22 disposed along core 20. Suture 40 may then be wrapped around and through the protrusions and perforations 22 in a manner conducive to increase the surface area of suture 40 that is exposed during coating as discussed in more detail below. In some embodiments, more than one protrusion may be disposed along core 220.

Suture 40 may be wrapped around spool 10 in a cross-wise pattern as illustrated in FIG. 1. Suture 40 may be arranged on spool 10 in such a manner as to allow for maximum suture surface area exposure. Suture 40 may also be arranged on spool 10 to expose a pre-determined amount or side of suture 40. Suture 40 may be wrapped around spool 10 in any configuration depending on the surface area exposure desired or length of time desired for exposure to solutions and/or compositions. Perforations 22 in spool 10 allow for coating composition 52 to penetrate the part of suture 40 lying closest to core 20 of spool 10. The wrap of suture 40 facilitates the subsequent spool dip operation of spool dip system 1.

With reference again to FIG. 1, spool dip system 1 for coating spool 10 of suture 40 is illustrated in accordance with the present disclosure. Spool 10 of suture 40 is placed into dip tank 50 including coating composition 52 and submerged under coating composition 52 for a period of time sufficient for coating composition 52 to coat suture 40. In embodiments, the soaking process may last from about 2 minutes to about 16 hours or more depending on the type of suture 40 and/or the type and concentration of an active agent used in coating composition 52.

In embodiments, the soaking process of the spool dip operation may take place under mild agitation. Agitation may be achieved by regular or intermittent motion of coating composition 52 within dip tank 50. Agitation may occur by imparting movement to dip tank 50 itself by, for example, rocking, vibrating, or shaking dip tank 50 or by imparting movement to the coating composition 52 contained within the dip tank 50 by rotating a blade or other stirring device within dip tank 50 or by a jet or stream of coating composition 52 circulating within dip tank 50.

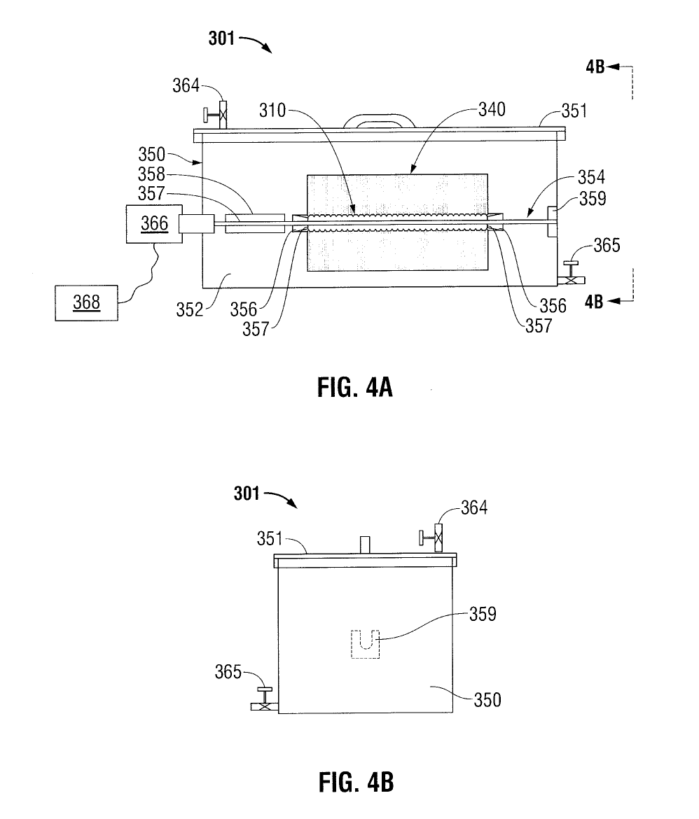

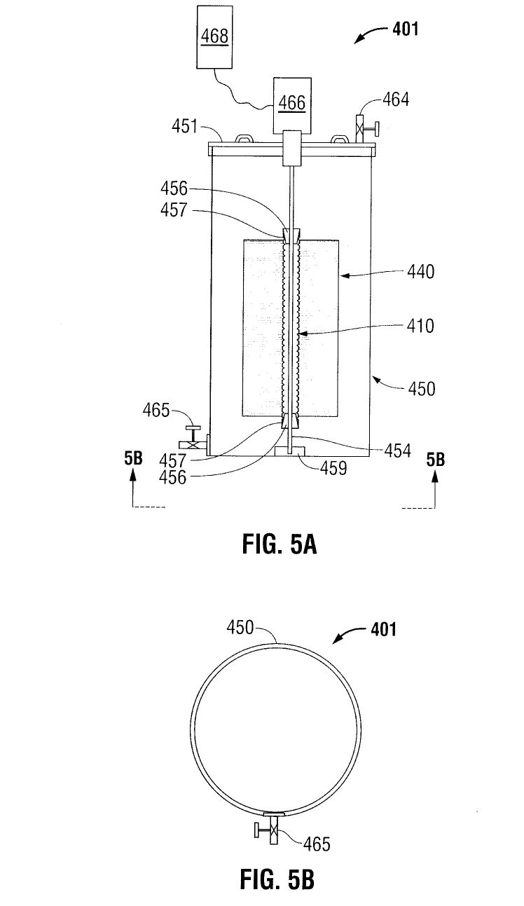

As illustrated in FIG. 3, table shaker 62 may be placed at the base of tank 50 to transmit vibrations to tank 50. Tank 50 is held in place with holding rods 63 of table shaker 62. Agitation may also be imparted by use of an external circulation pump or by rotating the mounted spool as illustrated in FIGS. 4A and 5A and described below. Spool 10 of suture 40 may be mounted on post 54 in dip tank 50 and movement is imparted to post 50, thus agitating spool 10 of suture 40. Other forms, speeds, and patterns of agitation are contemplated as appreciated by those skilled in the art.

At the end of the spool dip operation, spool 10 of suture 40 is separated from coating composition 52. Spool 10 may be either removed from dip tank 50 or dip tank 50 may be drained of coating composition 52.

Referring now to FIGS. 4A and 4B, there is illustrated horizontal tank 350 equipped with rotational shaft 354. Spool 310 of suture 340 may be mounted on shaft 354 and loaded into tank 350. Optionally, bearing block 359 may be utilized to provide support for rotational shaft 354. Spool 310 is securely positioned on shaft 354 via use of spool cone adapters 356 and shaft connecting adapter 358 for length adjustment. Lock screws 357 may be used to secure spool cone adapters 356 and shaft connecting adapter 358 about spool 310.

Coating compositions 352 may then fill tank 350 to a desired level through vent/fill valve 364 or the open top of tank 350 when tank cover 351 is removed. After valve 364 is closed and/or tank cover 351 replaced, rotational driver, such as motor 366, is started at a desired speed for a predetermined period of time to spin spool 310 within coating composition 352. Controller 368 controls motor 366 and provides rotations per minute (RPM) control of motor 366. It is envisioned that tank 350 may include more than one shaft 354 to couple with a corresponding numbers of spools 310 and that the shafts 354 may be controlled by the same or individual rotational drivers. It is also contemplated that more than one spool 310 may be placed on a single shaft 354.

After soaking is complete, tank 350 may be drained of coating composition 352 via valve 365 and spool 310 may be spun on rotational shaft 354 at a predetermined speed for a predetermined amount of time to remove excess coating composition 352 from spool 310. Spool 310 may then be dried at room temperature with or without inert gas or air sweeping. Conversely, spool 310 may be dried in an oven at a set temperature and humidity level or by vacuum drying under reduced pressure. In embodiments utilizing sweeping, air is introduced from drain valve 365 while vent valve 364 is open. Tank cover 351 may remain on tank 350 or tank 350 may be placed under a vent hood with tank cover 351 removed. In embodiments in which elevated temperatures are desired, hot air or hot gas, such as N₂, may be used. In embodiments, spool 310 may be rotating on shaft 354 during sweeping.

In some embodiments, a second coating composition may be introduced into tank 350 after sweeping/drying. Wetting, spinning, and drying of spool 310 may be repeated multiple times within tank 350 to coat suture 340 with any subsequent coating compositions by use of fill valve 364 and drain valve 365.

FIGS. 5A and 5B illustrate an alternative embodiment of the integrated coating and drying tank of FIGS. 4A and 4B. Like components are similarly numbered as those illustrated in FIGS. 4A and 4B and only the differences will be described below. In the current embodiment, tank 450 is cylindrical with alternate placement of fill/vent and drain valves 464 and 465 to allow for vertical orientation of spool 410.

Coating composition 52 maintained in dip tank 50 may include an active agent, but any coating composition useful for coating medical devices may be applied to medical devices using the present system and method. Coating composition 52 may be a solution, dispersion, or emulsion including, for example, one or more polymeric materials and/or one or more bioactive agents.

The coating composition may include active agents, such as drugs and/or polymer drugs, bioactive agents, and combinations thereof, as well as non-active agents. Polymer drugs may include biocompatible polymers, including polymers that are non-toxic, non-inflammatory, chemically inert, and substantially non-immunogenic in the applied amounts. Examples include anti-inflammatories, such as NSAIDS, antibiotics, antioxidants, and chemotherapy drugs.

Coating composition 52 may include organic or aqueous solvents in which the active and non-active agents as well as other compounds are dissolved or combined to form coating composition 52. Organic solvents include, but are not limited to, acetone, isopropyl alcohol, other alcohols, alkanes, methylene chloride, other chlorinated solvents, and combinations thereof. These solvents are capable of being removed from the coated suture 40 through the drying operations as will be discussed below.

Coating composition 52 may also include surfactants to increase the wettability of the coating composition 52 on suture 40. Surfactants include, but are not limited to, anionic surfactants such as sodium stearate, sodium cetylsulfate, polyoxyethylene laurylether phosphate, and sodium N-acyl glutamate; cationic surfactants such as stearyldimethylbenzylammonium chloride and stearyltrimethylammonium chloride; amphoteric (amphipathic/amphophilic) surfactants such as alkylaminoethylglycine hydrochloride solutions and lecithin; and non-ionic surfactants such as glycerin monostearate, sorbitan monostearate, sucrose fatty acid esters, propylene glycol monostearate, polyoxyethylene oleylether, polyethylene glycol monostearate, polyoxyethylene sorbitan monopalmitate, polyoxyethylene polyoxypropylene glycol, polyoxyethylene castor oil, polyoxyethylene lanolin, as well as poloxamer, polyethylene glycol, and polyethylene oxide derivatives, and combinations thereof.

In embodiments, coating composition 52 may be an antimicrobial colonization coating solution which is a combination of compounds, wherein the active agent is one or more antimicrobial agents. Suitable antimicrobial agents include triclosan, also known as 2,4,4′-trichloro-2′-hydroxydiphenyl ether; chlorhexidine and its salts, including chlorhexidine acetate, chlorhexidine gluconate, chlorhexidine hydrochloride, and chlorhexidine sulfate; silver and its salts, including silver acetate, silver benzoate, silver carbonate, silver citrate, silver iodate, silver iodide, silver lactate, silver laurate, silver nitrate, silver oxide, silver palmitate, silver protein, and silver sulfadiazine; polymyxin; tetracycline; aminoglycosides; such as tobramycin and gentamicin; rifampicin; bacitracin; neomycin; chloramphenicol; miconazole; quinolones such as oxolinic acid, norfloxacin, nalidixic acid, pefloxacin, enoxacin and ciprofloxacin; penicillins such as oxacillin and pipracil; nonoxynol 9; fusidic acid; cephalosporins, and combinations thereof. In addition, antimicrobial proteins and peptides such as bovine lactoferrin and lactoferricin B and antimicrobial polysaccharides such as fucans and derivatives may be used as antimicrobial agents in the coating of the present disclosure. Biomolecules such as heparin, fibrin, fibrinogen, cellulose, starch, and collagen are typically also suitable as an antimicrobial component.

In embodiments, the antimicrobial agents may be diluted. The antimicrobial compound or mixture of compounds thereof, may be about 0.01 percent by weight to about 3 percent by weight. In embodiments, about 0.1 percent by weight to about 1 percent by weight. Any concentration may be chosen to reduce the concentration gradient of solutes from the outermost to innermost wraps of suture 40 on spool 10 in order to achieve maximum uniformity throughout suture 40.

In another embodiment, coating composition 52 may contain one or more bioactive agents. The term “bioactive agent,” as used herein, is used in its broadest sense and includes any substance or mixture of substances that have clinical use. Consequently, bioactive agents may or may not have pharmacological activity per se, e.g., a dye. Alternatively, a bioactive agent could be any agent which provides a therapeutic or prophylactic effect, a compound that affects or participates in tissue growth, cell differentiation, a compound that may be able to invoke a biological action such as an immune response, or could play any other role in one or more biological processes.

Examples of classes of bioactive agents which may be utilized in accordance with the present disclosure include, antimicrobials; analgesics; antipyretics; anesthetics; antiepileptics; antihistamines; anti-inflammatories; cardiovascular drugs; diagnostic agents; sympathomimetics; cholinomimetics; antimuscarinics; antispasmodics, hormones; growth factors; muscle relaxants; adrenergic neuron blockers; antineoplastics; immunogenic agents; immunosuppressants; gastrointestinal drugs; diuretics; steroids; lipids; lipopolysaccharides; polysaccharides; enzymes; non-steroidal antifertility agents; parasympathomimetic agents; psychotherapeutic agents; tranquilizers; decongestants; sedative hypnotics; steroids; sulfonamides; sympathomimetic agents; vaccines; vitamins; antimalarials; anti-migraine agents; anti-parkinson agents such as L-dopa; anti-spasmodics; anticholinergic agents (e.g., oxybutynin); antitussives; bronchodilators; cardiovascular agents such as coronary vasodilators and nitroglycerin; alkaloids; analgesics; narcotics such as codeine, dihydrocodeinone, meperidine, morphine, and the like; non-narcotics such as salicylates, aspirin, acetaminophen, d-proxyphene and the like; opoid receptor antagonists such as naltrexone and naloxone; anti-cancer agents; anti-convulsants; anti-emetics; antihistimines; anti-inflammatory agents such as hormonal agents, hydrocortisone, prednisolone, prednisone, non-hormonal agents, allopurinol, indomethacin, phenylbutzone and the like; prostaglandins and cytotoxic drugs; estrogens; antibacterials; antibiotics; anti-fungals; anti-virals; anticoagulants; anticonvulsants; antidepressants; antihistamines; and immunological agents. It is also intended that combinations of bioactive agents may be used.

Other examples of suitable bioactive agents which may be included in the coating composition include viruses and cells; peptides; polypeptides and proteins; analogs; bacteriophages; muteins and active fragments thereof, such as immunoglobulins, antibodies, and cytokines (e.g., lymphokines, monokines, chemokines); blood clotting factors; hemopoietic factors; interleukins (IL-2, IL-3, IL-4, IL-6); interferons (β-IFN, (α-IFN and γ-IFN)); erythropoietin; nucleases; tumor necrosis factor; colony stimulating factors (e.g., GCSF, GM-CSF, MCSF); insulin; anti-tumor agents and tumor suppressors; blood proteins; gonadotropins (e.g., FSH, LH, CG, etc.); hormones and hormone analogs (e.g., growth hormone); vaccines (e.g., tumoral, bacterial and viral antigens); somatostatin; antigens; blood coagulation factors; growth factors (e.g., nerve growth factor, insulin-like growth factor); protein inhibitors, protein antagonists, and protein agonists; nucleic acids, such as antisense molecules, DNA and RNA; oligonucleotides; polynucleotides; and ribozymes.

In embodiments, coating composition 52 may contain one or more non-active agents. Non-active agents include polymers and combination of polymers. Examples of non-active agents include hyaluronic acid, carboxymethyl cellulose, polyvinyl pyrrolidones, polyvinyl alcohols, polyethylene glycol, polyethylene oxides, polypropylene glycol, polypropylene oxides, polytribolate, polyglycolide, polylactide, caprolactone, polybutylene adipate, phospholipids, pospholipid polymers, silicone, their copolymers and/or block polymers, and combinations thereof. Non-active agents may also include fatty acid components that contain a fatty acid, a fatty acid salt, or a salt of a fatty acid ester. Suitable fatty acids may be saturated or unsaturated, and include higher fatty acids having more than about 12 carbon atoms. Suitable saturated fatty acids include, for example stearic acid, palmitic acid, myristic acid, and lauric acid. Suitable unsaturated fatty acids include oleic acid, linoleic acid, and linolenic acid. In addition, an ester of fatty acids, such as sorbitan tristearate or hydrogenated castor oil, may be used. Suitable fatty acid salts may include the polyvalent metal ion salts of C6 and higher fatty acids, particularly those having from about 12 to about 22 carbon atoms, and mixtures thereof. Fatty acid salts including the calcium, magnesium, barium, aluminum, and zinc salts of stearic, palmitic, and oleic acids may be useful in some embodiments of the present disclosure. Particularly useful salts include commercial “food grade” calcium stearate which consists of a mixture of about one-third C16 and two-thirds C18 fatty acids, with small amounts of the C14 and C22 fatty acids.

Suitable salts of fatty acid esters may also be included in the coating compositions applied in accordance with the present disclosure. Calcium silicate and calcium stearoyl lactylate may be used, alone or in combination, with other non-active ingredients listed above. Salt of a lactylate ester of a C10 or greater fatty acid may also be used and include: calcium, magnesium, aluminum, barium, or zinc stearoyl lactylate; calcium, magnesium, aluminum, barium, or zinc palmityl lactylate; calcium, magnesium, aluminum, barium, or zinc olelyl lactylate; with calcium stearoyl-2-lactylate (such as the calcium stearoyl-2-lactylate commercially available under tradename VERV from American Ingredients Co., Kansas City, Mo.) being particularly useful. Other fatty acid ester salts which may be utilized include those selected from the group consisting of: lithium stearoyl lactylate, potassium stearoyl lactylate, rubidium stearoyl lactylate, cesium stearoyl lactylate, francium stearoyl lactylate, sodium palmityl lactylate, lithium palmityl lactylate, potassium palmityl lactylate, rubidium palmityl lactylate, cesium palmityl lactylate, francium palmityl lactylate, sodium olelyl lactylate, lithium olelyl lactylate, potassium olelyl lactylate, rubidium olelyl lactylate, cesium olelyl lactylate, and francium olelyl lactylate.

Coating composition 52 may also include furanones, such as halogenated furanones, brominated furanones, or other quorum sensing interrupters. Furanones, including halogenated furanones and/or hydroxyl furanones, are known as inhibitors of quorum sensing. Quorum sensing, also known as bacterial signaling, is recognized as a general mechanism for gene regulation in many bacteria, and it allows bacteria to perform in unison such activities as bioluminescence, swarming, biofilm formation, production of proteolytic enzymes, synthesis of antibiotics, development of genetic competence, plasmid conjugal transfer, and spoliation. Furanones, including halogenated and/or hydroxyl furanones, may block quorum sensing and inhibit the biofilm formation of bacteria in amounts that are substantially less harmful to mammalian cells. Given their mechanism of action, furanones' antipathogenic properties may be effective against a broad spectrum of infectious agents and may be able to reduce and/or prevent colonization of both gram positive and gram negative bacteria, including those noted above.

After spool 10 of suture 40 is sufficiently coated, the wet spool 10 of suture 40 is then dried. Spool 10 of suture 40 is spun dry to remove excess coating composition 52. Spool 10 of suture 40 may be spun on any mechanical device that imparts rotational movement to spool 10. In embodiments, spool 10 of suture 40 may be spun on post 54 in dip tank 50 after coating composition 52 is drained. In embodiments, spool 10 is spun horizontally to uniformly remove excess coating composition 52. Alternative axes of spinning are possible depending on the orientation of suture 40 on spool 10.

Spinning may last from about 2 minutes to about 8 hours depending on the amount of suture 40 on spool 10 and the speed, temperature, and humidity at which spinning occurs. In embodiments, spool 10 is spun at about 30 rpm to about 120 rpm. In some embodiments, spool 10 is spun at about 45 rpm to about 70 rpm. In other embodiments, spinning is performed with proper ventilation at room temperature or slightly elevated temperatures thereof. Spinning may occur with or without gas stripping. Gas stripping may be utilized to remove alcohols or other volatile solvents used in coating composition 52. During gas stripping, a dry and/or warm carrier gas is passed over suture 40 in order to remove alcohols or other volatile solvents. The gas may be air or inert gases, such as, for example, nitrogen, carbon dioxide, and the like.

In embodiments, spool 10 may be dried further in a vacuum drying chamber. The vacuum drying chamber dries suture 10 at elevated temperatures ranging from a low of about room temperature of 25° C. and up to a high of about 100° C.

The spool 10 of suture 40, once coated and dried, may be stored in a dry room as coating composition 52 has formed an antimicrobial pre-coat on suture 40 which has penetrated the suture structure. The crevices between individual fibers of suture 40 have been filled with the antimicrobial component or compounds which eliminate the potential sites available for microbial colonization.

Pre-coated suture 40 may be coated with second coating composition 53. Second coating composition 53, and any other subsequent coating composition(s), may consist of active or non-active agents as described above for first coating composition 52. Further, second coating composition 53 may also include other components, such as bioactive agents and solvents, as discussed above and combinations thereof. Second coating composition 53 may be the same or different from first coating composition 52.

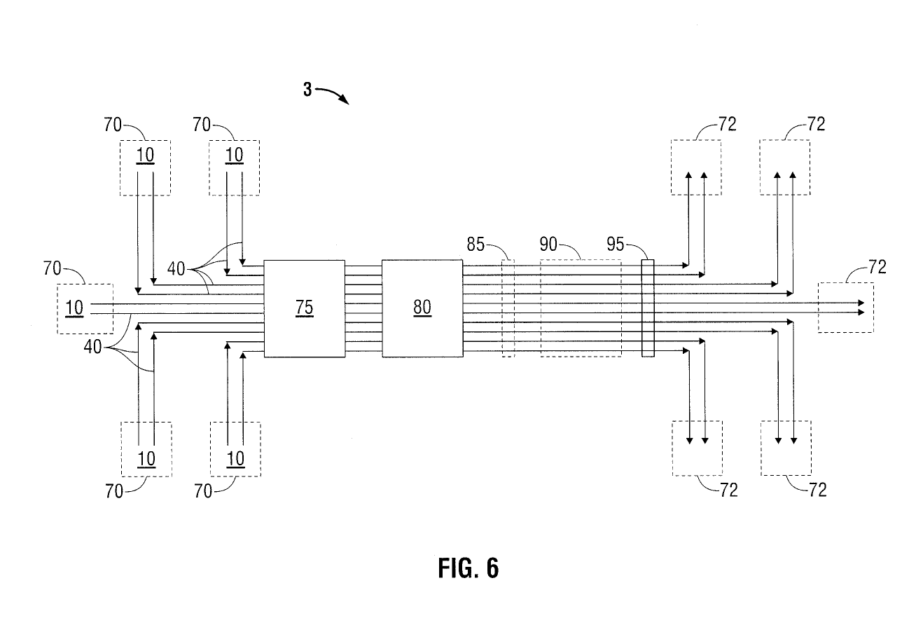

In embodiments, second coating composition 53 may be disposed in dip tank 50 wherein spool 10 of suture 40 may be subjected to a spool dip operation as was illustrated in spool dip system 1 of FIG. 1 with use of first coating composition 52 or in the integrated coating and drying systems illustrated in FIGS. 4 and 5. Alternatively, suture 40 may be subjected to a different coating device or process. In embodiments, suture 40 may be unwound from spool 10 and subjected to a suture line coating process or non-contact dip coating system, such as the horizontal dip coating system 3 as illustrated in FIG. 6 in accordance with the present disclosure.

FIG. 6 illustrates a schematic (or wire) diagram for a horizontal dip coating system 3 for coating one or more sutures 40 simultaneously. Horizontal dip coating system 3 includes pay-off winder 70 and dip coater 80. At least one line of suture 40 from at least one spool 10 is passed through dip coater 80 via pay-off winder 70. Pay-off winder 70 unwinds suture 40 from spool 10 and feeds suture 40 into dip coater 80.

The incoming line of suture 40 may pass through calendering apparatus 75 to facilitate penetration of coating composition 53 into the interstices of suture 40, especially when horizontal dip coating system 3 is used to apply a second or subsequent coating composition 53 to suture 40. Generally, a braided suture 40 is passed between two cylindrical calendering rollers, each having a smooth surface. The rollers are arranged substantially parallel to each other, but may be transverse to the axial orientation of suture 40. A mechanical compression force is applied to suture 40 by the rollers so that suture 40 is compressed radially inward and expands laterally in a transverse direction. Additionally, or alternatively, suture 40 may be compressed in a different or opposite direction than that stated above.

Dip coater 80 may contain at least one coating applicator such as, a coating tube, v-shaped notch, or other mechanism filled with second coating composition 53. The line of suture 40 is passed through, and immersed in, second coating composition 53 in the coating station of dip coater 80 before exiting.

The exiting line of suture 40 may optionally pass air wiper 85 which may be configured to blow gas, such as air or inert gases, on passing suture 40 in order to remove any excess coating composition 53. Dryer 90 may be positioned thereafter. Dryer 90 may be set to a temperature that is dependent on coating composition 53 used. It may range from about ambient room temperature of 25° C. up to about 100° C. Dryer 90 may also use a heated gas to dry suture 40. Optionally, air cooler 95 may be configured to blow cold air on suture 40 to cool the dried suture 40. Suture 40 may then be re-wound by use of take-up winder 72.

While FIG. 6 illustrates a typical suture line coating system, any suture coating device, system or method may be used to perform a second, third, or any subsequent coating as within the purview of those skilled in the art. Second coating composition 53 may be coated on suture 40 with any applicator within the purview of those skilled in the art, such as by dipping, spraying, drip coating, use of coating/filling heads and the like. For example, suture 40 may be coated by passing suture 40 under tension into a dip tank then through a drying tunnel. Suture 40 may be coated by use of a syringe to drip coat coating composition 53 on suture 40 while it is moving. Coating and/or filling heads may also be used to coat coating composition 53 on suture 40 as suture 40 is passed through a filling head applicator.

Drying is performed substantially immediately after applying second coating composition 53 in order to remove solvents from coated suture 40 as well as ensure that none of coating composition 53 is wiped away or removed from suture 40 by contact with other materials. Drying may include heating, vacuum drying, air drying, and/or air or inert gas stripping or combinations thereof as described above. Once dry, suture 40 may be re-spooled.

In embodiments, suture 40 may be coated with a third or more additional or subsequent coating compositions using the same or different coating compositions as first and second coating compositions 52 and 53, as well as the same or different coating device, applicator, system and/or methods.

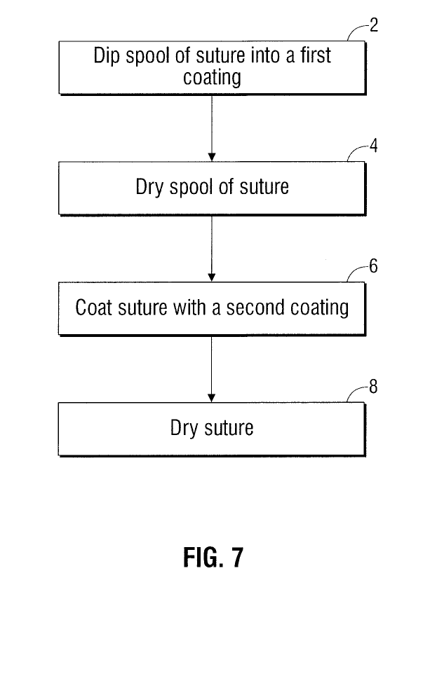

Referring now to the block diagram of FIG. 7, a spool dip and overcoat process is illustrated for coating suture 40 in accordance with the principles of the present disclosure. In step 2, a spool dip step, spool 10 of suture 40 is placed within dip tank 50 including first coating composition 52. Suture 40 soaks in coating composition 52, optionally with mild agitation, in order for coating composition 52 to impart a pre-coating to suture 40 on spool 10 and to maintain coating composition uniformity. Suture 40 is then dried in step 4. Suture 40 is dried by spinning spool 10. Optionally, drying step 4 may also include heating, vacuum drying, air drying, and/or air or inert gas stripping or combinations thereof. Suture 40 may then be coated with a second coating composition 53 as shown in step 6. Second coating composition 53 may be applied to suture 40 as described in step 2 by use of spool dip system 1, or second coating composition 53 may be applied via a different coating device and/or applicator, such as horizontal dip coating system 3. Suture 40 is then dried as stated in step 8 via a dryer or drying chamber through heating, vacuum drying, air drying, and/or air or inert gas stripping or combinations thereof.

It will be understood that various modifications may be made to the embodiments disclosed herein. Therefore, the above description should not be construed as limiting, but merely as an exemplification of preferred embodiments. Those skilled in the art will envision other modifications within the scope and spirit of the present disclosure. Such modifications and variations are intended to come within the scope of the following claims.

What is claimed is:

1. A system for coating sutures comprising:
a spool including a core having a suture wrapped thereabout;
a dip tank including a first coating composition, the dip tank configured to submerge the spool therein thereby coating the suture with the first composition to form a pre-coated suture; and
a coating device including a second coating composition, the coating device configured to overcoat the pre-coated suture.

2. The system of claim 1, wherein the core of the spool is perforated.

3. The system of claim 1, wherein the suture is cross-wound around the core.

4. The system of claim 1, wherein the core includes flanged ends.

5. The system of claim 4, wherein the flanged ends of the core are perforated.

6. The system of claim 1, wherein the first coating composition includes an active agent.

7. The system of claim 1, wherein the second coating composition is the same as the first coating composition.

8. The system of claim 1, wherein the dip tank includes a post configured and dimensioned to retain the spool in a submerged position within the dip tank.

9. The system of claim 1, wherein the coating device is selected from the group consisting of a dip tank, a horizontal dip coater, a coating head, a filling head, a sprayer, and a dip coat syringe.

10. A method of coating a suture comprising:
providing a spool of suture including a core having a suture wrapped thereabout;
placing the spool in a dip tank filled with a first coating composition thereby forming a pre-coated suture on the spool;
drying the spool of suture; and
coating the pre-coated suture with at least a second coating composition to form an overcoat on the pre-coated suture.

11. The method of claim 10, wherein placing the spool of suture into the first coating composition further comprises soaking the suture in the first coating composition from about 2 minutes to about 16 hours.

12. The method of claim 11, wherein soaking the suture in the first coating composition further comprises agitating the spool of suture in the first coating composition.

13. The method of claim 10, wherein the first coating composition includes an active agent.

14. The method of claim 10, further comprising draining the first coating composition from the dip tank.

15. The method of claim 10, wherein coating the pre-coated suture further comprises passing the suture through a horizontal suture line coating device.

16. A coated suture produced by the method of claim 10.

17. An integrated suture coating and drying tank system comprising:
at least one spool including a core having a suture wrapped thereabout; and
a tank configured to receive a coating composition, the tank including at least one rotational driver coupled to at least one shaft, the at least one shaft configured to removably couple to the at least one spool, wherein the at least one rotational driver is configured to spin the at least one shaft and the at least one spool within the tank in the presence and in the absence of the coating composition to respectively form a coating on the suture and dry the suture.

18. The integrated suture coating and drying tank system of claim 17, wherein the tank includes a first valve and a second valve, wherein the first valve is configured to introduce the coating composition into the tank and the second valve is configured to remove the coating composition from the tank.

19. The integrated suture coating and drying tank system of claim 18, wherein the second valve is configured to introduce a gas into the tank for drying the suture and the first valve is configured to remove the gas from the tank.

20. The integrated suture coating and drying tank system of claim 18, wherein the first valve is adapted to introduce a subsequent coating composition into the tank to form a subsequent coating on the suture.
